(19) United States
(12) Patent Application Publication
(10) Pub. No.: US 2012/0179122 A1
(43) Pub. Date: Jul. 12, 2012

(54) DEVICE FOR APPLYING AN OPHTHALMIC MEDICAMENT MIST

(75) Inventors: Eran Eilat, Herzliya (IL); Robert David, Atlanta, GA (US); Joshua Altman, Tel Aviv (IL); Moshe Tshuva, Tel Aviv (IL)

(73) Assignee: ADVANCED OPHTHALMIC PHARMA LTD, Tel Aviv (IL)

(21) Appl. No.: 13/384,842

(22) PCT Filed: Jul. 21, 2010
(86) PCT No.: PCT/IL10/00582

§ 371 Date: Apr. 3, 2012

Related U.S. Application Data
(60) Provisional application No. 61/227,456, filed on Jul. 22, 2009.

(51) Int. Cl. A61F 9/00; A61M 35/00

(52) U.S. Cl. 604/290; 604/296; 604/291

(57) ABSTRACT

A device for applying an ophthalmologic medicament to a patient's eye is disclosed. The device comprises spectacles defined by a frame and at least one lens; at least one container adapted for accommodating the ophthalmologic medicament; the container is incorporated into the frame; dosing mechanism adapted to control doses of the medicament; and at least one nozzle in a fluid interconnection with the medicament container; the nozzle is secured mechanically to the frame; the nozzle is adapted for providing a medicament mist flow. The nozzle is placed in front of a peripheral portion of a patient's eye slot and adapted to provide the mist flow in front of the patient's eye.

### FIELD OF THE INVENTION

The present invention relates to a device and methods for applying an ophthalmic medicament mist, and, more specifically, to a device providing a side flow of a mist containing a preservative-free ophthalmic medicament.

### BACKGROUND OF THE INVENTION

Treating the human eye, and in particular applying medicine to the eyeball, is a sensitive and often difficult task. The most common form of drug delivery is by topical application of the medicament to the eye's surface, e.g., by drops. The eye is uniquely suited to surface administration because drugs can penetrate through the cornea, reach therapeutic concentration levels inside the eye, and exert their beneficial effects there. In fact, ninety to ninety-five percent of drugs delivered to the eye are currently administered through eye drops. Rarely, drugs for the eye are administered orally or by injection, either because they reach the eye in too low a concentration to have the desired pharmacological effect, or because their use is complicated by significant systemic side effects.

Topical eye drops, though effective, have several drawbacks. When an eye drop is instilled in the eye, it overfills the conjunctival sac, the pocket between the eye and the eyelid, causing a substantial portion of the drop to be lost due to overflow from the lid margin onto the cheek. In addition, a substantial portion of the drop remaining on the ocular surface is drained into the nasolacrimal duct, thereby reducing availability of the drug to be absorbed. Not only is this portion of the drug dose lost before it can cross the cornea, but this excess drug is carried into the nose and throat where it may be absorbed into the local or general circulation, leading to systemic side effects, sometimes serious.

To compound the problems described above, patients often do not use their eye drops as prescribed. Often, this poor compliance is due to an initial stinging or burning sensation caused by the eye drop. Certainly, instilling eye drops in one's own eye can be difficult, in part because of the normal reflex to protect the eye. Therefore, sometimes one or more drops miss the eye. Older patients may have additional problems instilling drops due to arthritis, unsteadiness, and decreased vision, and pediatric and psychiatric patient populations pose difficulties as well. Bottled eye medication needs refrigeration after being opened, hence the cold drops add to the discomfort. Finally, most currently used formulations in ophthalmology contain preservatives, mainly Benzalkonium Chloride (BAK), a substance known to be toxic to the cornea.

There have been several developments to assist patients in applying medication to the eye. U.S. Pat. No. 6,610,036 provides an eye drop dispensing device that includes a trough member with a space to receive a cartridge housing enclosing a collapsible bag containing an ophthalmic liquid and comprising a spring finger applying pressure to the bag. It allows for spring-powered pump to spray a predetermined quantity of an ophthalmic liquid into a user's eye.

U.S. Pat. No. 6,336,917 provides an eye mist apparatus for treatment of an eye having an outer housing that contacts the bony orbit surrounding the eye. An inner housing, concentrically disposed within the outer housing contacts the eyelid, pushing the eyelid back, exposing the eye. One end of the inner housing is adapted to receive a dispenser for administering a metered spray of medicine, powdered or liquid, or lavage to the eye as it is held open by the ocular treatment apparatus. The dispenser nozzle is equipped with a contrasting mark, which provides a target for the eye to focus on ensuring the eye is optimally positioned to receive the metered spray. The spray is described as being directed straight onto the anterior surface of the eyeball, so that none of the medication is wasted. However, it is possible that delivery directly onto the eyeball surface may cause blinking reflex and discomfort to some users. US Patent Application Publication 2005/0255144 provides articles and methods for drug delivery by way of hydrogel for treatment of dry eye conditions. It allows for passive transference of drug from a dilute solution into the hydrogel. The hydrogel, when placed in contact with the eye, delivers the drug. The delivery of the drug is sustained over an extended period of time.

US Patent Application Publication 2006/0020253 provides an implantable device including a body defining a reservoir, and a head provided at the end of the body and closing the reservoir. The head is made from a permeable material adapted to release the medication from the reservoir to an implant site at a determinable rate.

U.S. Pat. No. 5,893,515 provides a device for creating and applying a mist, vapor, or spray from a liquid. The device contains a spinning rotor within a mist chamber. The rotor has inner walls that taper conically outwardly from the open bottom of the rotor to a hole near the top of the rotor. Liquid is pumped by a finger-actuated pump from a cartridge module into a bowl surrounding the bottom end of the rotor. A spray is created as the liquid is formed into droplets as it passes through the hole in the rotor under centrifugal force.

US Patent Application Publication 2002/0124843 provides an apparatus and a method for delivering medicine for one or more eyes. The apparatus includes goggles worn around the eyes. An atomizer nebulizes medicine into a medicine carrying fog such that the fog discharges from the fog outlets to deliver medicine to one or both eyes.

U.S. Pat. No. 6,962,151 discloses a nebulizer for atomizing liquid solutions (i.e. converting to aerosol). The nebulizer includes an aerosol generator that atomizes the liquid through a vibrating diaphragm into particle sizes that are efficiently delivered to the treated area. This nebulizer is currently commercialized under the trade name eFlow®. Classic jet and ultrasonic nebulizers have the disadvantage of potentially denaturizing the active agent by high shear forces (jet and ultrasonic nebs) and temperature increase (ultrasonic nebs). eFlow® incorporates a “gentle” aerosolization mechanism that minimizes exposure of the drug to shear stresses by reducing the shear stresses and the residence time in the shear fields and does not heat the liquid formulation. U.S. Pat. Nos. 5,152,456; 5,261,601, and 5,518,179 disclose further aspects of the eFlow® technology.

PCT Application WO2006/082588 describes a device for ophthalmic administration of pharmaceutical ingredients configured to direct a mist of the pharmaceutical composition at the eye. In this application, there is recognition of the need for maintaining sterility of the device. This is achieved by sterilizing the skin contact surface with the device between individual administrations. As stated therein, a device that is quick and easy to sterilize allows high throughput and safe ophthalmic administration of a pharmaceutical composition, for example in a hospital or clinic where many patients may be treated with one device, or in high-throughput situations, for example when it is desired to treat a population for an epidemic or endemic condition or to inoculate or immunize a population. One embodiment incorporates a self-sterilization device, in the form of an element radiating the contact surface of the device. However, in this application, other than by the assumed use of preservatives, no consideration appears to have been made of the need to ensure that the pharmaceutical composition itself remains sterile, especially in the typical domestic setting of prolonged sequential use of the device by a single person.

Most dispensers consist of a nozzle, i.e., an internally incorporated mechanism providing the dispensed fluid its flowing characteristics. A few dispensers which do not comprise a traditional nozzle (i.e., Venturi nozzle dispensers) were suggested in the art. Thus, a small number of T-type Flow Nozzles were disclosed in the literature: e.g., JP60101434A2: Hot Water Supplying Apparatus and JP2001145450A2: Auxiliary Tool For Spraying On High Plate present mechanisms for spraying fluids.

WO 2007/132446-discloses a device for applying a medicament to the eye, consisting of a single or pair of eye goggles, into which a fine mist or fog of the medicament is sprayed. The medicament is contained in a canister attached to an aperture in the goggle or goggles, and the mist is generated either by the configuration of the outlet valve, or by a baffle disposed within the goggle volume opposite the outlet valve. In accordance with the technical solution taught in WO 2007/132446, the mist flow is directed straight from the nozzle to the patient's eye, such that the mist flow traumatizes the patient's eye:

Thus, there remains an unmet and long-felt need to provide an apparatus for ophthalmic treatment that is configured for creating a cloud of the medicament mist originated from the side.

### SUMMARY OF THE INVENTION

It is hence one object of the invention to disclose a goggle-like dispenser of ophthalmologic medicaments, comprising: a single goggle held over the eye and having a seal to prevent escape of the medicament mist. The goggle-like dispenser potentially comprises a wing portion adapted to be folded over the eyecup in between uses to protect the eyecup from contamination from the outside environment. The device is possibly provided with a mechanism for storing and delivering preservative-free medicaments. The device is further provided as fully mechanical (without electrical components). The goggle-like dispenser dispenses a steady mist consisting of droplet sizes on the range of approximately less than about 50 microns that gently blanket and settle on the surface of the eye and that do not interfere with vision or irritate the eye. The device is potentially equipped with a control mechanism that can be altered to control the direction, velocity, size, shape and exposure time of the droplets to effectively deliver the drug to the eye. The device is also useful for a single-action application.

It is another object of the invention to disclose a goggle-like dispenser of ophthalmologic medicaments, comprising: a single goggle held over the eye, comprising a seal to prevent escape of the medicament mist within a confined volume surrounding the patient's eye; a mechanism for storing and mechanically delivering preservative-free medicaments; and a mechanism to dispense a steady mist consisting of droplet sizes on the range of approximately less than about 50 microns that gently blanket and settle on the surface of the eye and that do not interfere with vision or irritate the eye.

It is another object of the invention to disclose the goggle-like dispenser as defined above, wherein the dispenser further comprises at least one container adapted for accommodating ophthalmologic medicaments; at least one nozzle placed in front of a peripheral portion of a patient's eye slot, provided in a fluid interconnection with the container; the nozzle mechanically secured to the dispenser body and adapted to provide the medicament mist flow. The nozzle is adapted to laterally provide the mist flow within the confined volume in a direct and-non-eye-impinging manner.

It is another object of the invention to disclose the goggle-like dispenser as defined above, wherein the at least one container is incorporated within the frame or within the lens.

It is another object of the invention to disclose the goggle-like dispenser as defined above, wherein the nozzle is mechanically secured to the frame or to the lens.

It is another object of the invention to disclose the goggle-like dispenser as defined above, wherein the medicament is accommodated in the container in either a highly pressurized nitrogen-free state or non-pressurized manner.

It is another object of the invention to disclose the goggle-like dispenser as defined above, wherein the medicament mist is preservative-free.

It is another object of the invention to disclose the goggle-like dispenser as defined above, wherein the dispenser further comprises a control mechanism that can be altered to control the direction, velocity, size, shape and exposure time of the droplets to effectively deliver the ophthalmologic medicaments to the eye. The control mechanism is potentially provided with a one-way dosing valve. Alternatively or additionally, the control mechanism regulates or meters one or more fluid's parameters selected from a group consisting of dispensing-time protocol, droplet volume, fluid flux, droplet weight, batch unit, and any combination thereof.

It is another object of the invention to disclose the goggle-like dispenser as defined above, wherein the medicament mist is dispensed in such a manner that it remains in a dispersed state for a preset time scale, the time scale is between about 0.5 sec to about 5 sec, between about 5 sec to about 40 sec, between about 10 to about 50 sec, or between about 15 to about 60 sec.

It is another object of the invention to disclose the goggle-like dispenser as defined above, wherein the dispenser further comprises a medicament heater and/or cooler. It is in the scope of the invention wherein the heater and or coolers are selected in a non-limiting manner, from a group consisting of Pelletier devices, ultrasonic emitters, Ohmic resistors, etc.

It is another object of the invention to disclose the goggle-like dispenser as defined above, wherein the at least one container is a plurality of microcontainers, in particular disposable microcontainers; and the microcontainers are individually connectible in fluid-connection with the nozzle. The microcontainers are optionally fed by a feeding mechanism selected from the group consisting of a revolving nosepiece, a cartridge, a magazine, a clip, and any combination thereof.

It is another object of the invention to disclose the goggle-like dispenser as defined above, wherein a connector is adapted to consecutively or concurrently open the microcontainers and feed corresponding doses of the ophthalmic medicament into the nozzles.

It is another object of the invention to disclose the goggle-like dispenser as defined above, wherein the dispenser further comprises at least one protective cover; adapted to be folded over the eyecup in between uses to protect the eyecup from contamination from the outside environment.

It is another object of the invention to disclose the goggle-like dispenser as defined above, wherein the protective cover is a form-fitting wing.

It is another object of the invention to disclose a method of dispensing ophthalmic medicaments. The method comprises providing a goggle-like dispenser of ophthalmologic medicaments, as defined in any of the above, filling the container with the ophthalmic medicament; placing the device on the patient; and, facilitating a flow of a mist or vapor medicament into the confined volume defined between the patient's eye and the lens; wherein the flowing is performed in a manner that gently blanket and settle on the surface of the eye and that do not interfere with vision or irritate the eye.

It is another object of the invention to disclose a goggle-like Venturi nozzle dispenser of ophthalmologic medicaments. The device comprises a single goggle held over the eye, comprises a seal to prevent escape of the medicament mist within a confined volume surrounding the patient's eye; a mechanism for storing and mechanically delivering preservative-free medicaments; and a mechanism to dispense a steady mist flow having an outlet, in a fluid connection with at least one Bernoulli-type junction of multiple W conduits, W is an integer equal to or bigger than two; the mist consisting of droplet sizes on the range of approximately less than about 50 microns that gently blanket and settle on the surface of the eye and that do not interfere with vision or irritate the eye. The term ‘about’ refers in the present invention to a value being ±20% of the defined measure.

It is another object of the invention to disclose the goggle-like Venturi nozzle dispenser as defined above, wherein the multiple W conduits provides a multiple conduits junction, the junction is selected from a group consisting of L-type, Y-type, T-type or and X-type.

It is another object of the invention to disclose the goggle-like Venturi nozzle dispenser as defined above, wherein the angle between the main longitudinal axis of two conduits is angle theta θ, and wherein θ is more than about 0°.

It is another object of the invention to disclose the goggle-like Venturi nozzle dispenser as defined above, wherein θ is about 90°, and especially wherein θ ranges between about 75° to about 85°.

It is another object of the invention to disclose the goggle-like Venturi nozzle dispenser as defined above, wherein the at least one container is incorporated within the frame or within the lens.

It is another object of the invention to disclose the goggle-like Venturi nozzle dispenser as defined above, wherein the fluid outlet is mechanically secured to the frame or to the lens.

It is another object of the invention to disclose the goggle-like Venturi nozzle dispenser as defined above, wherein the medicament is accommodated in the container in either a highly pressurized nitrogen-free state or non-pressurized manner.

It is another object of the invention to disclose the goggle-like Venturi nozzle dispenser as defined above, wherein the medicament mist is preservative-free.

It is another object of the invention to disclose the goggle-like Venturi nozzle dispenser as defined above, wherein the dispenser further comprises a control mechanism adapted to control doses of the medicament. The control mechanism is possibly provided with a one-way dosing valve.

It is another object of the invention to disclose the goggle-like Venturi nozzle dispenser as defined above, wherein the dosing is provided by one or more of a group consisting of predetermined time protocol, volume, flux, weight, batch unit, number of bursts, and any combination thereof.

It is another object of the invention to disclose the goggle-like Venturi nozzle dispenser as defined above, wherein the medicament mist is dispensed in such a manner that it remains non-condensable matter for a preset time scale, the time scale is between about 1 to about 5 sec, between 5 sec to about 40 sec, between about 10 to about 50 sec, or between about 15 to about 60 sec.

It is another object of the invention to disclose the goggle-like Venturi nozzle dispenser as defined above, wherein the dispenser further comprises a medicament heater and/or cooler.

It is another object of the invention to disclose the goggle-like Venturi nozzle dispenser as defined above, wherein the mechanism for storing of the medicaments is a plurality of microcontainers; the microcontainers are individually connectible in fluid-connection with the nozzle.

It is another object of the invention to disclose the goggle-like Venturi nozzle dispenser as defined above, wherein the dispenser further comprises at least one protective cover, especially a form-fitting wing; the protective cover permanently or temporarily attached to the lens or dispenser body by mechanical attachment such as a hinge or equivalent, and effectively forming a seal when closed.

It is another object of the invention to disclose a method of dispensing ophthalmic medicaments. The method comprises providing a goggle-like dispenser of ophthalmologic medicaments, as defined in any of the above; filling the container with the ophthalmic medicament; placing the device on the patient; and, facilitating a flow of a mist or vapor medicament into the confined volume defined between the patient's eye and the lens; wherein the flowing is performed in a manner that gently blanket and settle on the surface of the eye and that do not interfere with vision or irritate the eye.

It is another object of the invention to disclose a goggle-like dispenser of ophthalmologic medicaments. The aforesaid dispenser comprises goggle-like spectacles having a frame and at least one lens which define a confined volume surrounding patient's eye to be treated; at least one container incorporated within the spectacles, adapted for accommodating the medicament; at least one nozzle placed in front of a peripheral portion of a patient's eye slot, provided in a fluid interconnection with the container; the nozzle is mechanically secured to the spectacles and adapted to provide a medicament mist flow in both direct, i.e., non-baffled and non-mediated manner, and non-eye-impinging manner, i.e., in a non-perpendicular manner. It is well in the scope of the invention wherein nasal medicaments and ear medicaments are to be dispensed, and the peripheral portion of a patient to be treated is a nasal or era cavity, respectively.

The combination of direct and non-eye-impinging flow of the droplets mist ensures, inter alia, effective coating of the eye with the medicament, avoids overdose i.e., casual penetration of the medicament through the mucosal coating of the patient's nostrils and into the blood circulation, eye irritation, loss of expensive medicament etc., overcomes patient conscious and unconscious hesitation and restraint for dispensing the medicament towards his eye, thus increases both short- and long-term treatment efficiency, and lastly, because the hereby disclosed dispenser is designed to dispense aerosol in a non-baffled and non-mediated manner, it is adapted, according to some embodiments of the invention, to significantly avoid accumulation of dispensed medicaments on top of a baffle, thus eliminating secondary contamination of the confined volume (e.g., inoculum by microorganisms of the baffle which is continuously wet by the medicament) and ensuring safe reuse of the device.

The term ‘goggle-like spectacles’ refers hereinafter in a non-limiting manner to eye goggles, goggle-like members, spectacles, monocles, masks or the like of any size, shape, and design, which define a confined volume adjacent to one or both eyes to be treated.

Another object of the invention is to disclose at least one container unlimitedly incorporated within the frame and/or within at least one of the lenses. It is in the scope of the invention wherein the lens is transparent and allows the transmission of light, semi-transparent, or at least partially made of non-transparent materials.

It is in the scope of the invention wherein the goggle-like dispenser as defined hereinafter is at least partially made of polymers and plastics, such as polymethyl metaacrylates (PMMA, PHMMA etc.), polyamides such as nylons and the like, polyurethanes, polylactic acids, polyalkylenes, such as polyethylene of polyporopylene etc., glassware, cardboards, rubber, composite materials, metal-ware, or any mixture and combination thereof. It is also in the scope of the invention wherein the goggle-like dispenser or modules thereof are either disposable or provided for multi-use purposes.

A further object of the invention is to disclose the nozzle mechanically secured to the frame; e.g., in a lateral (side) portion of the frame in respect to the eye slot. A further object of the invention is to disclose the nozzle mechanically secured to the lens, especially in a lateral (side) portion of the lens in respect to the eye slot. The term ‘lateral’ refers hereinafter, in a non-limiting manner the side of the eye: a bottom side portion, a medial side portion, a top side portion, or any combination thereof.

A further object of the invention is to disclose the medicament accommodated in the container in either a highly pressurized nitrogen-free state (i.e., a pressure being above atmospheric conditions, such as 3 atm to 5 atm, 30 atm to 50 atm etc.) or non-pressurized manner. The term ‘non-pressurized manner’ refers hereinafter to any pressure less than ‘high pressure’ and includes also a moderate pressure. The pressure of the medicament in the container may be set as know in the art, namely, in a range of about 1 atm to about 5 atm, about 5 atm to about 50 atm, about 50 atm to about 150 atm etc.; the term ‘about’ refers hereinafter to a measure being ±25% of the defined value.

A further object of the invention is to disclose the goggle-like dispenser comprises a dosing mechanism or any other metering mechanism adapted to control doses of dispensed medicament. Hence for example, a metering spray pump design and/or a metering valve, e.g., a mechanism comprises a ball, knob or either static or actuated member, or other arrangement adapted to ensure proper metering of a desired quantity of an aerosol product from the valve assembly upon sufficient actuation or depression of the stem is utilizable. The dosing mechanism controls parameters of the dispensed aerosol, including, e.g., the dispensing time protocol (e.g, protocol of a sequence of a few bursts of mist), aerosol volume, flux, weight, batch unit, or any combination thereof.

A further object of the invention is to disclose the dosing mechanism provided with at least one one-way dosing valve.

A further object of the invention is to disclose the medicament mist dispensed in such a manner that it remains a non-condensable matter for a preset time scale. According to one embodiment of the invention, the time scale is provided in a non-limiting manner between about 1 to 5 sec or less, to about 40 sec, between about 10 to about 50 sec, or between about 15 to about 60 sec or more. It is in the scope of the invention wherein the mist comprises particles and/or droplets of average diameters ranging from nano-meter sizes (e.g., vapour or sub-micron size droplets) to micrometer sizes.

A further object of the invention is to disclose the goggle-like dispenser further comprises medicament and/or mist heater and/or cooler. It is in the scope of the invention wherein the at least one portion of the heating/cooling device (e.g., a Pelletier mechanism, a heating coil etc) is incorporated within the frame of the dispenser. The heating/cooling mechanism is either a direct mechanism (e.g., heating the dispensed fluid) or indirect (e.g., heating the lens).

A further object of the invention is to disclose the goggle-like dispenser which comprises a control unit. The control unit is adapted to control the flow and/or parameters of the medicament mist. It is in the scope of the invention wherein the control unit is patient sensitive (e.g., patient age, condition and degree of illness), medicament sensitive, protocol sensitive (e.g., programmable), operator sensitive (e.g., settable by either the user, by a nurse etc) or feedback-activated.

A further object of the invention is to disclose the goggle-like dispenser as defined in any of the above, wherein the container is provided as a set, array or a cartridge equipped with a plurality of microcontainers. The microcontainers are individually connectible in fluid-connection with the nozzle. It is in the scope of the invention wherein the microcontainers are fed towards the nozzle before dispensing by a feeding mechanism. This feeding mechanism is selected in a non-limiting manner from the group consisting of a revolving nosepiece, a cartridge, a magazine, a clip, and any combination thereof. It is in the scope of the invention where the term ‘microcontainer’ also refers to disposable microcontainers; to containers comprises micro-nozzles, to dispensing facilities comprises micro-Venturi-junctions, and micro-multi-phase-flow-mixing-chambers.

A further object of the invention is to disclose the goggle-like dispenser which comprises a connector which is adapted to consecutively open a preset portion of each of the microcontainers and feed corresponding preset dose of the ophthalmic medicament via the nozzles, into the confined volume.

It is also in the scope of the invention wherein multiple medicaments are utilized either simultaneously or separately, according to a preset protocol. Hence, the goggle-like dispenser as defined in any of the above potentially comprises multiple containers or a container with multiple separate sections, multiple nozzles, etc. It is also in the scope of the invention wherein one or more medicaments are dispensed by a biocompatible propellant, such as pressurized N₂.

A further object of the invention is to disclose a method of dispensing ophthalmic medicaments. The aforesaid method comprises steps selected in a non-limiting manner from providing a goggle-like dispenser as defined in any of the above; filling the container with at least one ophthalmic medicament; placing the device on the patient; blowing, jetting, or otherwise facilitating flow or otherwise jetting a mist flow in a lateral direction and containing the ophthalmic medicament into the confined volume, defined between the patient's eye and the lens. It is a core of the invention to provide a step or steps of blowing, jetting, or otherwise facilitating flow of the mist performed laterally (sideward direction in respect to the eye), such that the nozzle laterally provides the mist flow within the confined volume in both direct (i.e., non-baffled) and non-eye-impinging manner (i.e., in a non-perpendicular manner).

A further object of the invention is to disclose the dispensing method as defined above, wherein the method further comprises a step of incorporating at least one container within the frame and/or the lens.

A further object of the invention is to disclose the dispensing method as defined above, wherein the method further comprises a step of mechanically securing the nozzle to the frame.

A further object of the invention is to disclose the dispensing method as defined above, wherein the method further comprises a step of mechanically securing the container to the lens.

A further object of the invention is to disclose the dispensing method as defined above, wherein the method further comprises a step of accommodating the medicament within the container in a highly pressurized nitrogen-free state.

A further object of the invention is to disclose the dispensing method as defined above, wherein the method further comprises a step of blowing, jetting, or otherwise facilitating flow or otherwise jetting a preservative-free medicament mist.

A further object of the invention is to disclose the dispensing method as defined above, wherein the method further comprises a step of controlling the dispensed dosage.

A further object of the invention is to disclose the method as defined above, wherein the method further comprises a step of controlling the dispensed dosage performed by a one-way dosing valve.

A further object of the invention is to disclose the dispensing method as defined above, wherein the method further comprises a step of controlling the dispensed dosage characterized by one or more of a group consisting of predetermined time protocol, volume, flux, weight, batch unit, number or bursts, and any combination thereof.

A further object of the invention is to disclose the dispensing method as defined above, wherein the method further comprises a step of blowing, jetting, or otherwise facilitating flow of the medicament mist performed in such a manner that it remains non-condensable matter for a preset time scale, the time scale is between about 1 sec to about 5 sec, about 5 sec to about 40 sec, between about 10 sec to about 50 sec, or between about 15 sec to about 60 sec.

A further object of the invention is to disclose a method comprises steps of heating and/or cooling the medicament in either a direct or indirect manner.

A further object of the invention is to disclose the step of blowing, jetting, or otherwise facilitating flow wherein the step comprises, inter alia, a sub-step of controlling or otherwise regulating the flow and/or parameters of the medicament mist.

A further object of the invention is to disclose the sub-step of controlling the parameters performed according to at least one parameter, by selecting one or more members of a group consisting of time, droplets size and distribution, medicament type, flux, temperature and combination thereof.

A further object of the invention is to disclose the step of providing the dispenser. The method comprises inter alia a sub-step of providing microcontainers individually connectible in fluid-connection with the nozzle.

It is a further object of the invention to disclose a step of blowing, jetting, or otherwise facilitating flow of mist, wherein the method comprises, inter alia, sub-steps of feeding the microcontainers by a feeding mechanism, and selecting this feeding mechanism from the group consisting of a revolving nosepiece, a cartridge, a magazine, a clip and any combination thereof.

A further object of the invention is to disclose the method as defined above, wherein the method comprises a step of blowing, jetting, or otherwise facilitating flow of mist, and further comprises sub-steps of consecutively or concurrently opening the microcontainers and feeding corresponding doses of the ophthalmic medicament into the nozzles.

A further object of the invention is to disclose a goggle-like Venturi nozzle dispenser of ophthalmologic medicaments. The Venturi nozzle dispenser comprises: goggle-like spectacles having a frame and at least one lens which define a confined volume surrounding the patient's eye to be treated; at least one container incorporated within the spectacles, adapted for accommodating the medicament; at least one fluid outlet located in front of a peripheral portion of a patient's eye slot, provided in a fluid interconnection with the container; the outlet is a fluid connection with at least one Venturi-type junction of multiple W conduits, W being an integer equal to or greater than two and adapted to provide a medicament mist flow. The fluid's outlet, one or more, in this Venturi nozzle dispenser is preferably secured to the spectacles and adapted to provide a medicament mist flow in both direct, i.e., non-baffled and non-mediated manner, and non-eye-impinging manner, i.e., in a non-perpendicular manner.

Similarly, and in accordance to the defined above, a further object of the invention is to disclose a Venturi nozzle dispenser as defined above, wherein the combination of the Bernoulli-type junction and the outlet is adapted to laterally provide the mist flow within the confined volume in both direct and non-eye-impinging manner. A combination of direct and non-eye-impinging flow of the droplets mist solely via one or more fluid outlets (i.e., not via a nozzle) ensures, inter alia, effective coating of the eye with the medicament, avoids overdose i.e., casual penetration of the medicament through the nasolacrimal duct into the mucous coats of the patient's nasopharynx, eye irritation, loss of expensive medicament etc., overcomes patient conscious and unconscious hesitation and restraint for dispensing the medicament towards his eye, thus increasing both short- and long-term treatment efficiency, and lastly, because the hereby disclosed dispenser is designed to dispense aerosol in a non-baffled and non-mediated manner, significant accumulation of dispensed medicaments on top of a baffle is avoided, thus eliminating secondary contamination of the confined volume (e.g., inoculum by microorganisms of the baffle which is continuously wet by the medicament) and ensuring safe reuse of the device.

A further object of the invention is to disclose a Venturi nozzle dispenser as defined above, wherein the angle between the main longitudinal axis of two conduits is angle theta (θ), and wherein θ ranges between more than 0° to less than 180°, e.g., θ is about 90°, e.g., θ ranges between about 75° to about 85°.

A further object of the invention is to disclose a method of dispensing ophthalmic medicaments from a Venturi nozzle dispenser. The method comprises steps of providing a goggle-like Venturi nozzle dispenser; the dispenser comprises goggle-like spectacles having a frame and at least one lens which define a confined volume surrounding the patient's eye to be treated; at least one container incorporated within the spectacles, adapted for accommodating the medicament; at least one fluid outlet located in front of a peripheral portion of a patient's eye slot, provided in a fluid interconnection with the container; the outlet is fluid connection with at least one Venturi-type junction of multiple W conduits, W is an integer equal to greater than two and adapted to provide a medicament mist flow; preferably, the fluid outlet is adapted to provide the mist flow laterally within the confined volume in both direct and non-eye-impinging manner; filling the container with the ophthalmic medicament; placing the device on the patient; and by Bernoulli principle, blowing, jetting, or otherwise facilitating flow of a mist containing the ophthalmic medicament into the confined volume defined between the patient's eye and the lens.

A further object of the invention is to disclose a method as defined above, wherein the step of blowing, jetting, or otherwise facilitated flowing is performed laterally such that the outlet laterally provides the mist flow within the confined volume in both direct and non-eye-impinging manner.

A further object of the invention is to disclose a method as defined above, wherein the method further comprises a step of incorporating at least one container within the frame.

A further object of the invention is to disclose a method as defined above, wherein the method further comprises a step of incorporating at least one container within the lens.

A further object of the invention is to disclose a method as defined above, wherein the method further comprises a step of mechanically securing the fluid outlet to the frame.

A further object of the invention is to disclose a method as defined above, wherein the method further comprises a step of mechanically securing the fluid outlet to the lens.

A further object of the invention is to disclose a method as defined above, wherein the method further comprises a step of accommodating the medicament within the container in a highly pressurized nitrogen-free state.

A further object of the invention is to disclose a method as defined above, wherein the step of blowing, jetting, or otherwise facilitating flow of medicament mist is performed in a preservative-free manner.

A further object of the invention is to disclose a method as defined above, wherein the method further comprises a step of controlling the dispensed dosage.

A further object of the invention is to disclose a method as defined above, wherein the step of controlling the dispensed dosage is performed by a one-way dosing valve.

A further object of the invention is to disclose a method as defined above, wherein the step of controlling the dispensed dosage is characterized by one or more of a group consisting of predetermined time protocol, volume, flux, weight, batch unit, number of bursts, and any combination thereof.

A further object of the invention is to disclose a method as defined above, wherein the step of blowing, jetting, or otherwise facilitating flow of the medicament mist is performed in such a manner that it remains non-condensable matter for a preset time scale, the time scale is between about 1 sec to about 5 sec, between 5 sec to about 40 sec, between about 10 sec to about 50 sec, or between about 15 sec to about 60 sec.

A further object of the invention is to disclose a method as defined above, wherein the method further comprises the steps of heating and/or cooling the medicament.

A further object of the invention is to disclose a method as defined above, the step of blowing, jetting, or otherwise facilitating flow comprises a sub-step of controlling the flow and/or parameters of the medicament mist.

A further object of the invention is to disclose a method as defined above, wherein the sub-step of controlling the parameters is performed according to at least one parameter selected from the group consisting of time, droplets size and distribution, medicament type, flux, temperature and combination thereof.

A further object of the invention is to disclose a method as defined above, wherein the step of providing the dispenser comprises a sub-step of providing microcontainers individually connectible in fluid-connection with the nozzle.

A further object of the invention is to disclose a method as defined above, wherein the step of blowing, jetting, or otherwise facilitating flow of mist further comprises a sub-step of feeding the microcontainers by a feeding mechanism selected from the group consisting of a revolving nosepiece, a cartridge, a magazine, a clip, and any combination thereof.

A further object of the invention is to disclose a method as defined above, wherein the method further comprises the step of blowing, jetting, or otherwise facilitating flow of mist further comprises a sub-step of consecutively opening the microcontainers and feeding corresponding doses of the ophthalmic medicament into the nozzles. A further object of the invention is to disclose a method as defined above, comprising: providing a goggle-like dispenser of ophthalmologic medicaments, comprising a single goggle held over the eye comprising: a seal to prevent escape of the medicament mist within a confined volume surrounding the patient's eye; a mechanism for storing and mechanically delivering preservative-free medicaments; and a dispenser to dispense a steady mist consisting of droplet sizes on the range of approximately less than 50 microns; filling the container with the ophthalmic medicament; placing the device on the patient; and, facilitating a flow of a mist or vapor medicament into the confined volume defined between the patient's eye and the lens; wherein the facilitating is performed in a manner that gently blanket and settle on the surface of the eye and that do not interfere with vision or irritate the eye.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIG. 1a-c is a schematic view of the device for applying the ophthalmic medicament according to one embodiment of the invention;

FIG. 2 is a graph of the distribution of medicament aerosol particle sizes;

FIG. 3 is a schematic view of a device according to yet another embodiment of the invention;

FIG. 4 is a schematic view of the device provided with the microcontainers placed into a revolving nosepiece according to another embodiment of the invention;

FIG. 5a-b are isometric views and cross sections of the dispensing device adapted for dispensing the medicament from the microcontainer according to another embodiment of the invention;

FIG. 6a-e are isometric views and cross sections of the dispensing device adapted for dispensing the medicament from the microcontainer according to another embodiment of the invention;

FIG. 7a-b are schematic cross sections of a Venturi Y-type and T-type junctions according to another embodiment of the invention; and,

FIG. 8 is Venturi Y-type junction, according to another embodiment of the invention.

### DETAILED DESCRIPTION OF PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a device for applying an ophthalmologic medicament to a patient's eye.

The term ‘fog’ refers herein below to a cloud of droplets, 0.5-10 microns, characterized by a Freefall time (existence time per 1 inch free fall) of about 30 seconds or more; Terminal velocity 0-5 mm/sec and hazy appearance. The term ‘fine mist’ or ‘mist’ interchangeably refers herein below to a cloud of droplets, 20-50 microns, characterized by a Freefall time (existence time per minute) of about 1 to about 5 seconds; Terminal velocity 20-100 mm/sec. The visual appearance of such a mist is separate droplets. The term ‘less-fine mist’ refers herein below to a cloud of droplets, 50-150 microns, characterized by a Freefall time (existence time per minute) of about 0.1 to about 0.3 seconds; Terminal velocity 50-600 mm/sec. The visual appearance of such a mist is separate droplets. In practice, the terms fog and mist are interchangeable.

The terms ‘mist’ and ‘fog’ interchangeably and specifically further refer in the context of the present invention to a cloud of very small droplets provided with a Brownian motion, i.e., a cloud which is not under facilitated flow within a confined volume by any external mechanism, such as a fan or an injector. Moreover, ‘mist’ and ‘fog’ provided by the present invention is directed tangentially towards a confined volume adjacent to the eye, in a manner that droplets do not collide and thus decrease their mass and subsequently impinge the eye. The term ‘spray’ and ‘drizzle’ interchangeably refer herein below to a cloud of droplets, 200-500 microns, characterized by a Freefall time (existence time per minute) of about 0.05 seconds; Terminal velocity 2000 mm/sec. The visual appearance of such a spray or drizzle is droplets.

The momentum (kg*m/sec) of the cloud is about 1*10⁴ for spray, 1*10⁻⁵ for less fine mist, 10⁻⁵-1*10⁻⁶ for mist, and 1*10⁻⁸-1*10⁻⁹ for fog.

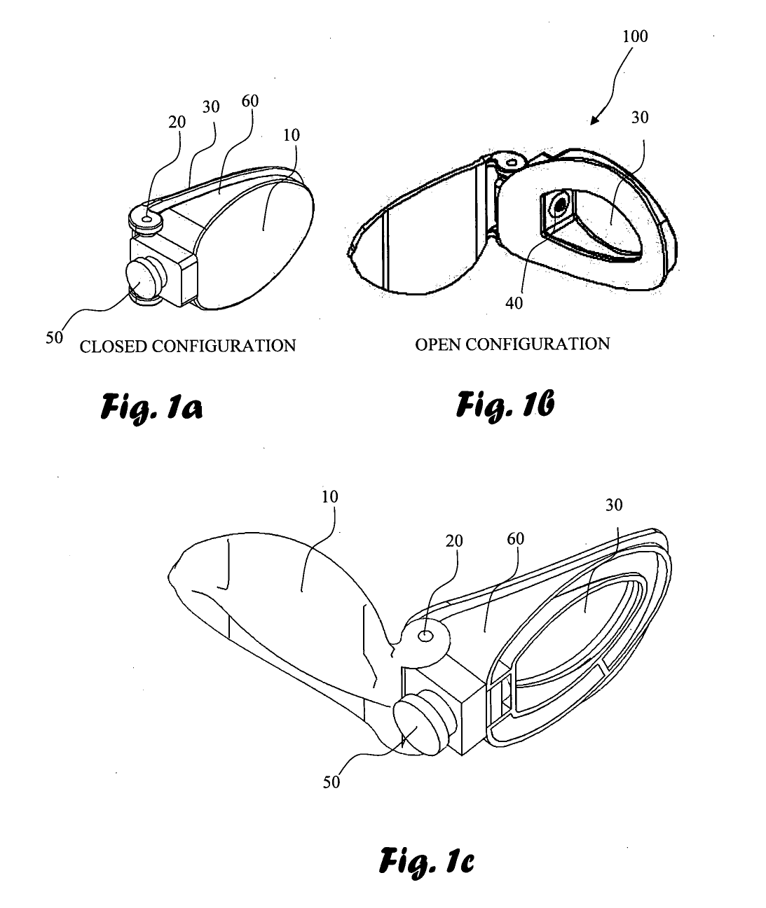

Reference is now made to FIG. 1a-c, illustrating in an out-of-scale manner general views of a device for dispensing an ophthalmic medicament 100, according to one embodiment of the invention. The goggle-like dispenser 100 of ophthalmologic medicaments presented herein its closed configuration (FIG. 1a) and its open (ready to use) configuration (rear view FIG. 1b, front view FIG. 1c) comprises a body 60 which comprises at least one lens 30 and optionally a frame which define a confined volume surrounding the patient's eye to be treated; at least one container (not shown) is adapted for accommodating the medicaments. At least one nozzle 40 is placed in front of a peripheral portion of a patient's eye slot, provided in a fluid interconnection with the container. The nozzle is mechanically secured to the dispenser body and is adapted to provide a medicament mist flow. The nozzle is further adapted to laterally provide the mist flow within the confined volume in a direct and-non-eye-impinging manner.

The terms ‘direct’ and ‘non-eye impinging’ relates, in a non-limiting manner, to passage of the droplets from the nozzle or the Venturi nozzle to the patient's eye in a direct manner, i.e., without pre-colliding on the surface of the dispenser, on the surface of an insert within the confined volume (e.g., fan-blades, a baffle, goggle-walls etc.). The terms also related to a facilitated flow of the cloud which is characterized by a minimal momentus is about 1*10⁻⁷ or 1*10⁻⁹ Kg*m/sec, e.g., about *10⁻⁸ Kg*m/sec. According to the specific embodiment disclosed in FIGS. 1a-c, the dispenser further comprises a wing 10, connected to the frame or the body of the dispenser by a hinge 20. Similarly, this embodiment further presents an optional single-handed dosing valve 50 of a single-action mechanism. It is in the scope of the invention wherein the wing is elastic or comprises semi-rigid rims, adapted to fit the contour of the opening of the goggle.

It is acknowledged in this respect that a dispenser with two goggle-like dispensers for left and right eyes, conjugated or otherwise incorporated by a mutual single frame is possible, according to yet a second embodiment of the invention.

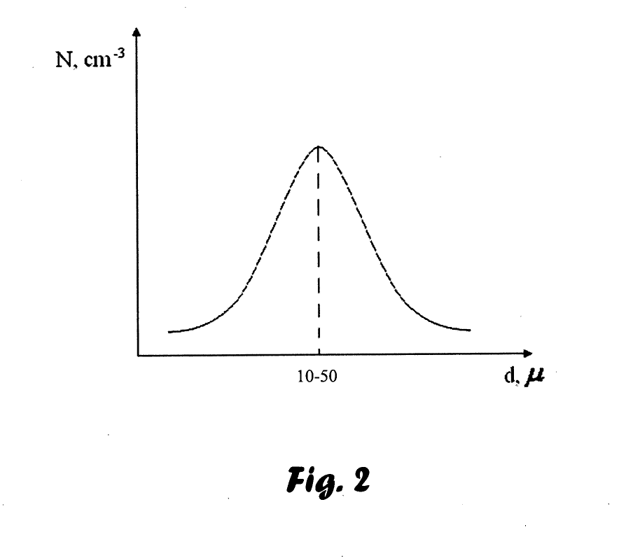

Reference is now made to FIG. 2, illustrating in a not-to-scale manner a graph of droplets distribution constituting dependence of relative quantity N of droplets in μm⁻³ on the size d of medicament droplets. As seen in FIG. 2, maximum value of droplets quantity corresponds to droplets of 10μ size. It is known that fog or mist, as defined and presented in the present invention, is described by the Gauss distribution.

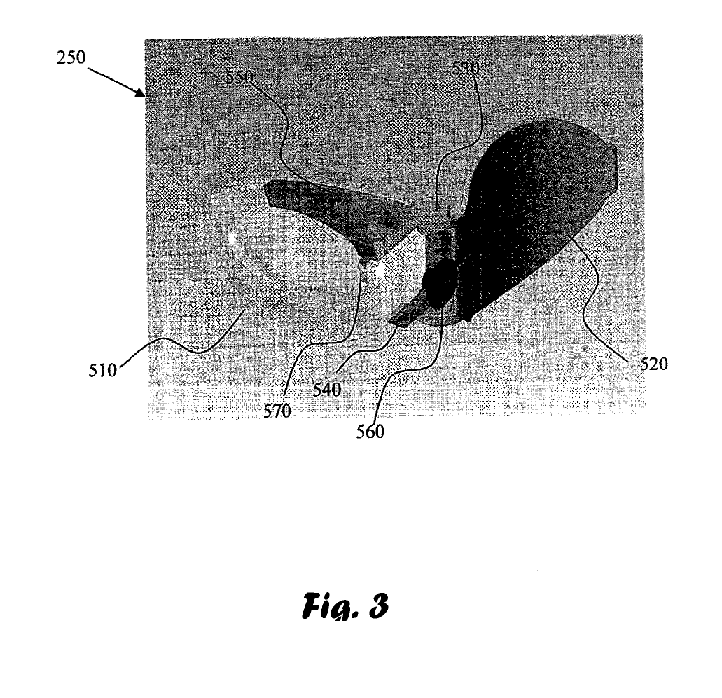

Reference is now made to FIG. 3, illustrating in a not-to-scale manner a 3-dimensional rendering of an alternative embodiment 250 of the device for applying an ophthalmic medicament. Goggle-like dispenser 250 comprises, inter alia, a lens cup 510, a protective cover 520 (a wing like member), a hinge 530, a control unit 540, a fluid reservoir 550, and actuator 560, optionally being a dosing valve, and a nozzle 570. Protective cover 520 fits snugly against lens 510 when closed to provide protection against e.g., dirt, bacteria, and foreign matter. After the user opens the device 250, the user holds lens cup 510 against his or her eye and presses actuator 560, which releases a metered dose of medicament through nozzle 570.

It is in the scope of the invention wherein container 550 retains the ophthalmic medicament that is pressurized up to 50 atm and does not contain nitrogen as a propellant gas. To isolate the ophthalmic medicament from the environment, nozzle 570 comprises a one-way dosing valve 560 that provides dispensing of the medicament and prevents environmental air from penetration into lens cup 510. Medicament's conduit 540 is controlled by the control unit 560 which is designed for dispensing the mist flow according to a predetermined time protocol and for recording the performed activity. The mist created is the central zone of the confined volume between the patient's eye and the goggle 510 is non-condensable mist to prevent the medicament from casual penetration through the mucosal coating of the patient's nostrils into the blood circulation.

It is in the scope of the invention wherein each, a portion or all of the following: the container, the medicament's conduit, the dosing valve and/or the nozzle are disposable modules, i.e., adapted to be replaced after their use.

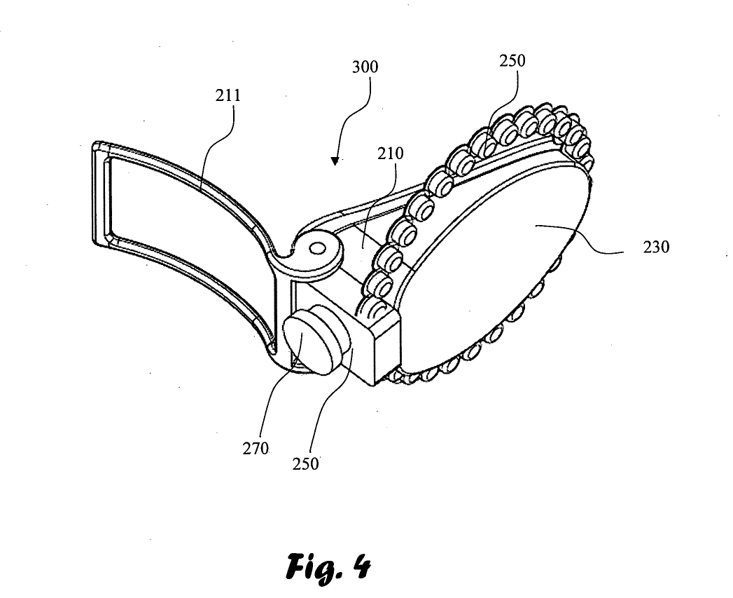

Reference is now made to FIG. 4, illustrating in an out-of-scale manner a schematic view of an alternative embodiment 300. The device 300 comprises a frame 210, lens 230, and protective wing 211. The device 300 is provided with a plurality of microcontainers 250 accommodating micro-doses of the ophthalmic medicament. The microcontainers 250 are unlimitedly placed into a feeding mechanism that enables the microcontainers 250 to be fed to a connector (not shown). This feeding mechanism is selected in a non-limiting manner from the group consisting of a revolving nosepiece, a cartridge, a magazine, a clip, and any combination thereof. The aforesaid connector is adapted to consecutively open the microcontainers 250 and feed the corresponding doses of the ophthalmic medicament into the nozzle (not shown). Microcontainers 250 accommodate the ophthalmic medicament which is pressurized up to 50 atm and does not contain nitrogen as a propellant gas. The dispensing is controlled by a dosing (controlling) unit 270 which is adapted for dispensing the mist flow according to a predetermined time protocol and for recording the performed activity. The mist created in the central zone of the volume between the patient's eye and the lens 230 is non-condensable mist to prevent the medicament from casual penetration through mucosal coating of the patient's nostrils into the blood circulation.

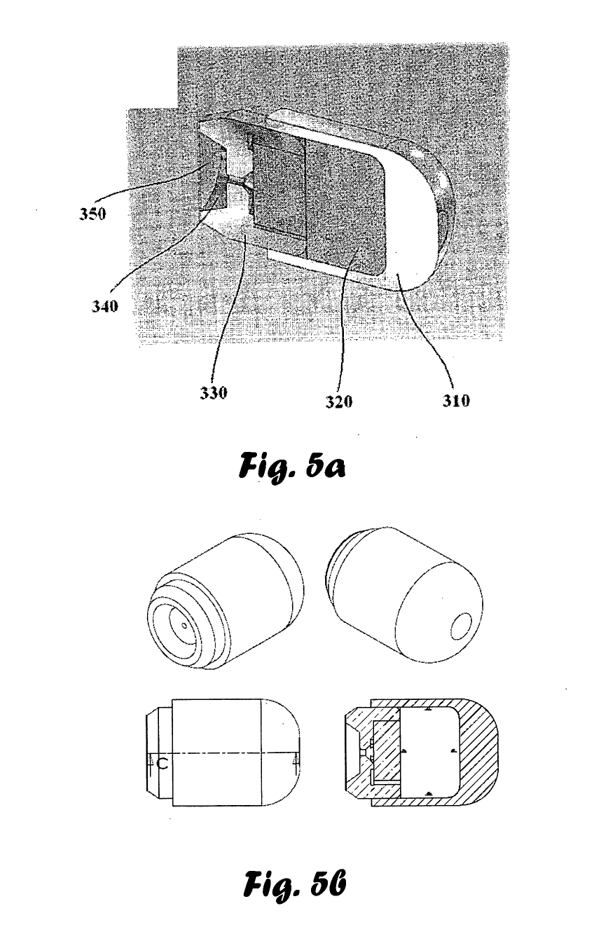

Reference is now made to FIG. 5a-b, illustrating in an out-of-scale manner a microcapsule 320. A process of mist generation is facilitated by nitrogen accommodated in a cavity 340. A nozzle 330 pricks a foil 350 and the medicament from the microcapsule 320 exits into the confined volume between the patient's eye and the device (not shown).

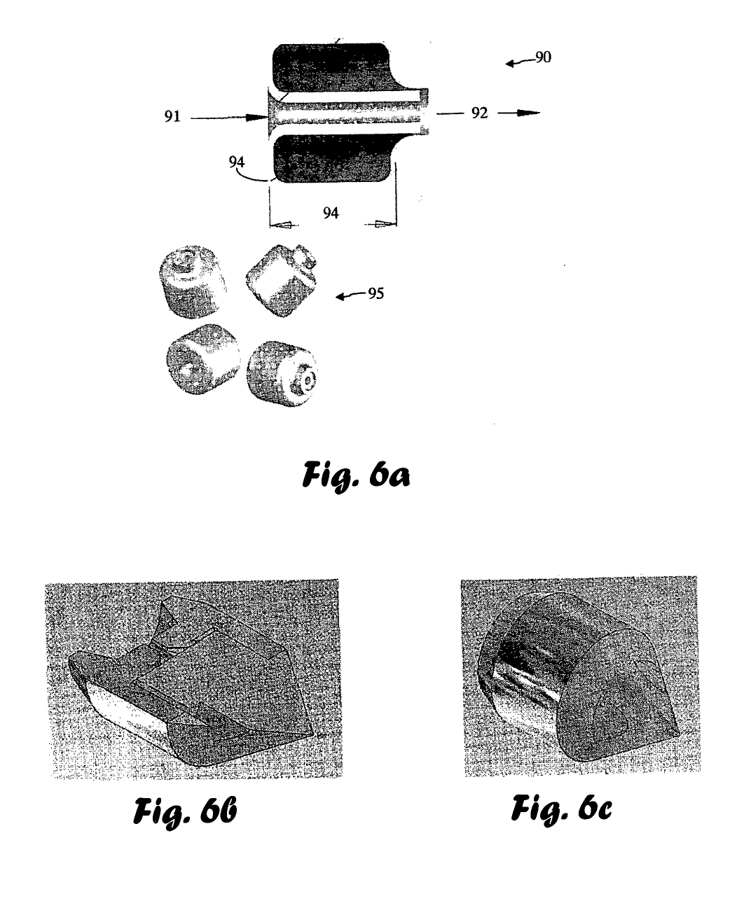

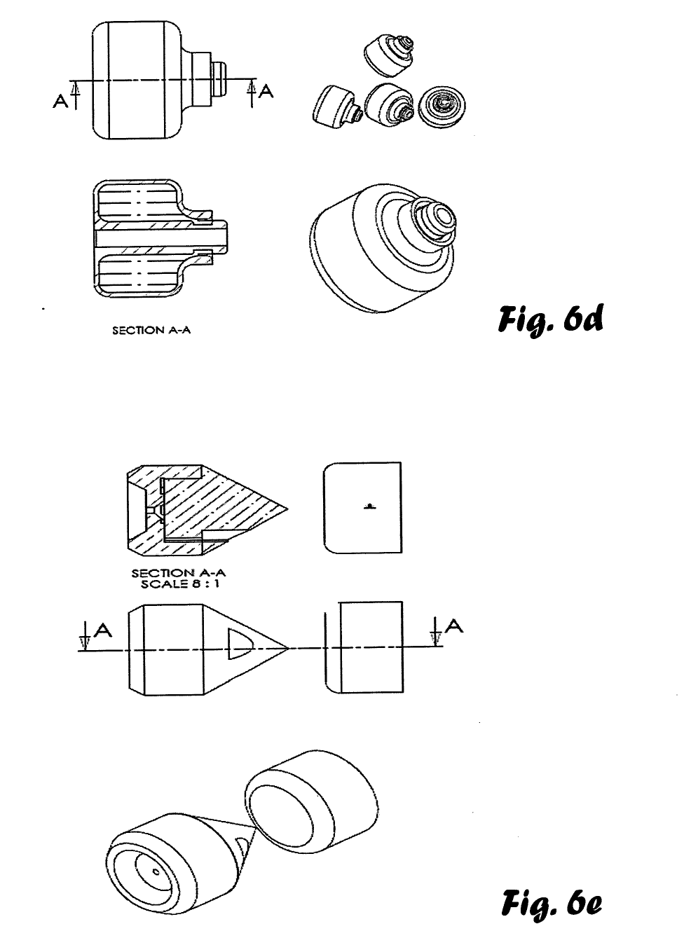

Reference is now made to FIG. 6a, presenting [upper portion] an out-of-scale cross section of a micro-container (90) according to one embodiment of the invention. This small (e.g., dimensions of about 5 to about 10 mm, for example, length 94 of about 7 mm), preferably yet not exclusively disposable canister, comprises fluid inlet (91) and jet outlet (92) dispensing a medication contained within volume 93. FIG. 6a further presents [lower portion] an out-of-scale perspective view of a micro-container (95) providing useful as, e.g., disposable Venturi-bullets. FIG. 6b and FIG. 6c presents cross sections of a piercing nozzle device, and a perspective view of the same, respectively. FIG. 6d-e present more illustrations of the same.

The present invention also discloses a goggle-like Venturi nozzle dispenser of ophthalmologic medicaments. The device comprises, inter alia, a body with at least one lens and optionally a frame which define a confined volume surrounding the patient's eye to be treated; at least one container adapted for accommodating the medicaments; and at least one fluid outlet located in front of a peripheral portion of a patient's eye slot, provided in a fluid interconnection with the container; the outlet is a fluid connection with at least one Bernoulli-type junction of multiple W conduits, W is an integer equal to or bigger than two and adapted to provide a medicament mist flow. The combination of the Venturi-type junction and the outlet is possibly adapted to laterally provide the mist flow within the confined volume in a direct and non-eye-impinging manner. The multiple W conduits potentially provides a multiple conduits junction, the junction is selected from a group consisting of L-type, Y-type, T-type or and X-type.

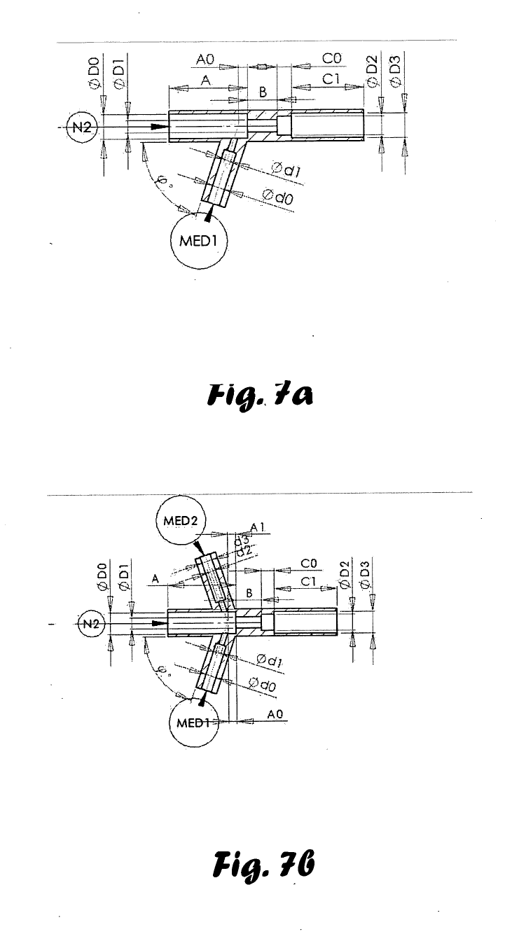

Reference is now made to FIG. 7a and FIG. 7b, illustrating in a non-limiting manner cross sections of two types of dispensers disclosed in the invention, namely a Y-type (i.e., one liquid medicament phase and one gaseous carrier phase, either in a symmetric or asymmetric arrangement) and a double-Y-type (X-type) (i.e., two liquid medicament phases and a single gaseous carrier phase) Venturi nozzle system. Dimensions of the inner sections of the Venturi nozzle systems are listed in the figures. Table 1 presents various, but not all possible dimensions:

TABLE 1
Junction's characteristics of Y-type and X-type Venturi nozzle
systems according to some embodiments of the invention (DEG, mm)

| Type | f | D0 | D1 | d0, d1 | D2, d3 | A1 | D1 | D2 | D3 | A0 | A | B | C0 | C1 |
|---|---|---|---|---|---|---|---|---|---|---|---|---|---|---|
| Y type | 0-90 | 0.3-3 | 0.2-2 | 0.2-2 | NA | NA | 0.2-2 | 0.2-2 | 0.3-3 | ±5 | 1-40 | 0-10 | 0-10 | 0-5 |
| T type | 90 | 0.3-3 | 0.2-2 | 0.2-2 | NA | NA | 0.2-2 | 0.2-2 | 0.3-3 | ±5 | 1-40 | 0-10 | 0-10 | 0-5 |
| L type | 0-90 | 0.3-3 | 0.2-2 | 0.2-2 | NA | NA | 0.2-2 | NA | NA | ±5 | 1-40 | 0-10 | 0 | 0 |
| X-type | 0-90 | 0.3-3 | 0.2-2 | 0.2-2 | 0.2-2 | ±5 | 0.2-2 | 0.2-2 | 0.3-3 | ±5 | 1-40 | 0-10 | 0-10 | 0-5 |

According to another embodiment of the invention, a Venturi nozzle dispensing apparatus is disclosed. The Venturi nozzle dispensing apparatus utilizes both Bernoulli's principle, which states that for an in viscid flow, an increase in the speed of the fluid occurs simultaneously with a decrease in pressure or a decrease in the fluid's potential energy; and Venturi effect which states a reduction in fluid pressure that results when a fluid flows through a constricted section of pipe: The fluid velocity increases through the constriction to satisfy the equation of continuity, while its pressure decreases due to conservation of energy: the gain in kinetic energy is balanced by a drop in pressure or a pressure gradient force. It is acknowledged in this respect that in a Venturi-type device, as disclosed in accordance with some embodiments of the present invention, fluid's flow rate and kinetic energy of the fluid (i.e., within the conduits of the device), altogether with parameters related to the behavior of condensate liquid within ambient conditions (outside the device), provided at the dispensed droplets to brake to smaller droplets thus to increase medical efficiency.

More specifically, the present invention discloses a Venturi nozzle dispenser of ophthalmologic medicaments. In particular, the present invention discloses a Venturi nozzle T-type or L-type conduit junctions, and a Venturi nozzle (i.e., T-type or L-type conduit arrangements which do not essentially containing a nozzle mechanism) facilitated-aerosol-jetting mechanism for drugs and preservative-free medicaments application. The term ‘preservative free’ relates, according to one embodiment of the invention, to the absence of benzalkonium chloride (i.e., alkyldimethylbenzylammonium chloride, ADBAC, or similar compounds) and its equivalents, and any other preserving substances, stabilizers, or similar compounds. The dispenser comprises, inter alia, enveloping mechanism (e.g., a frame, goggle-like envelope etc) to define a confined volume surrounding at least a portion (e.g., sphere, hemisphere, crescent-shaped hemisphere, etc.) of a patient's eye to be treated. The dispenser also comprises at least one container accommodating a single- or multi-component medicament, raw materials or processors thereof. Moreover, the dispenser comprises an L-type, Y-type or T-type Venturi nozzle dispensing mechanism, i.e., a junction of multiple W conduits, where W is an integer equal to or greater than two. A two-conduit junction refers hereinafter to T-type or alternatively L-type or Y-type, a three-conduit junction refers hereinafter X-type, and so on and so forth. It is in the scope of the invention wherein the aforesaid W conduits and junctions thereof apply the Bernoulli principle defined above. The Venturi nozzle apparatus is placed in front of a peripheral portion of a patient's eye slot, provided in a fluid interconnection with the container. The Venturi nozzle apparatus is preferably mechanically secured to the enveloping mechanism and adapted to provide an effective medicament mist flow. It is acknowledged in this respect that devices characterized by an X-type junction and any other multiple junctions are especially useful for admixing two or more medicaments or carriers to provide enhance therapeutic composition applied as a fine mist.

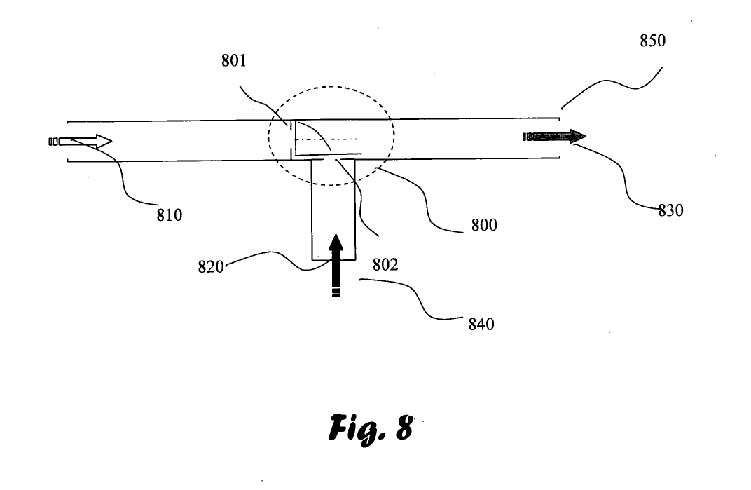

Reference is now made to FIG. 8, which is a schematic of a Venturi T-Junction. Gas flow 810 passes through orifice 801 and over orifice 802. This creates a pressure drop in zone 800, making it negative relative to tube 840, as represented by the graph. This causes fluid flow 820. Fluid passes through the orifice and mixed flow 830 emerges from the nozzle 850. The rate of fluid extracted from tube 840 depends on the instantaneous pressure difference across orifice 802. The total amount can be predicted based on the initial pressure difference, allowing for effective metering of a dosage.

Within the broken circle 800, one sees the pressure (Y axis) plotted against the distance from orifice 801 (X axis). As the distance increases, pressure decreases to a value lower that pressure in tube 820, such that suction is induced causing a predetermined metered dose of correctly size liquid droplets to be expressed from the outlet.

It is acknowledged in this respect that controlled droplet size is an important parameter in dispensation of ophthalmic medicament. In a directed stream, droplets should be large enough to travel in a straight path. In a fog, droplets that are too large will fall due to gravity and the fog will dissipate. Droplet size also directly affects droplet momentum, which contributes to the possibility of eye impingement. In the context of the present invention, the term ‘eye impingement’ or ‘impinging’ interchangeably refer according to one aspect of the invention, to droplets directly sticking the eye possessed of a momentum greater than imparted by Brownian motion alone.

The goggle-like dispenser of the present invention controls droplet direction and thus effectively delivers the drug to the eye and reduces the chances of impinging the eye. The dispenser provides very low mass droplets with very short ballistic trajectories at velocities which produce droplets of low momentum. Such low momentum droplets of high surface-to-volume ratios are designed to transfer very low impact to the eye and deliver maximum dosage.

The present invention enables dispensing of controlled dose and controlled exposure time of the eye to the dispensed fluid. The disclosed technology provides, however, a single-action application which actuates both mist production and ejection, and is an “idiot proof” operating system. Moreover, the unpleasant experience of having a medication applied directly to the eye is minimized due to the gentleness of the application and the brief duration of the procedure (seconds). The goggle-like dispenser enables control of exposure time through droplet size, velocity and realizing time. Via controlling the exposure time, one controls the absorption of the active compound into the eye. The goggle-like dispenser of the present invention discloses and enables an exposure time measure in seconds.

The present invention discloses a dispensing mechanism wherein the mist is transferred from the outlet to the confined volume in a direct manner, namely, without utilizing baffles, fan blades or suspenders, which tend to be contaminated and to collide small droplets to bigger droplets which eventually impinge the eye. The presented technology thus directs a steady mist gently blanketing and settling on the surface of the eye. A peripheral portion is targeted at a non-perpendicular angle so that the mist does not interfere with vision or irritate the eye.

The container 10 is unlimitedly incorporated into the spectacle frame 60. It is well within the scope of the invention wherein the container or two or more connected or non-connected containers are integrated within the frame, e.g., in at least a portion of the perimeter of the goggle' lens; above the frame, below the frame, in the side of the frame, adjacent the frame or remotely from the frame. A possible control unit 20 enables the patient to regulate a protocol and characteristics of the mist dispensed towards the eye. The regulation of the control unit is provided either manually or automatically.

The goggle-like dispenser of the present invention comprises, according to one embodiment of the invention, a container which is fully integrated within the frame. Possibly, the goggle-like dispenser is essentially tubeless. Moreover, it is in the scope of the goggle-like dispenser is potentially characterized by a dispenser which consists of a disposable nozzle-less Venturi, avoiding disadvantages of a detachable un-pressurized container, e.g., a container that is situated on the bridge of the spectacles; jetting cartridge like a jet-ink cartridge; and devices having long narrow tubes which emerge from the cartridge and partially snake around the circumference of each spectacle, such long and narrow tubes are inherently prone to kinking, blocking tearing and contamination; a removable (i.e., not integrated) cartridge with a refillable basin.

It is in the scope of the invention wherein the nozzle and/or nozzle-less Venturi are positioned in or on the goggle frame in one or more locations selected from a group consisting of the rim of the goggle, top the goggle, side the goggle, bottom the goggle, inside the goggle or otherwise within the lens of the goggle, or any combination thereof.

The present invention also discloses a goggle-like dispenser having anti-microbial properties. The dispenser is capable of safely storing and delivering preservative-free medicaments. The goggle-like dispenser incorporates novel safety features as follows: Pressure gradient—Bacteria generally cannot move against a pressure gradient. In AOP such a gradient is facilitated along the whole pathway, between the fluid container and the outlet until it is discharged. It is in the scope of the invention wherein at least a one first and at least one second compressed nitrogen (or CO₂ or air) containers are utilized, e.g., for flushing portions of the dispensed medicaments and for providing a steady and continuous pressure gradient along the medicament tubing. Alternatively or additionally, unidirectional and/or non-return valves are utilized to facilitated a pressure gradient which its lower pressure is located adjacent the eye to be treated.

Nitrogen Buffering—the tubing is possibly flushed with nitrogen before and/or after injection. It is in the scope of the invention wherein flushing is provided in a mechanism defined above which comprises, inter alia, N₂ one or more containers and/or microcontainers. One way valves—the current discloser teaches a potential use of no-return valves placed at each fluid communication point to prevent transmission of airborne microorganisms even if the pressure gradient were to be reversed. Disposable nozzles—the goggle-like dispenser potentially comprises a nozzle less Venturi outlets that are adapted to be replaced by the user after each use.

The goggle-like dispenser of the present invention seals the eye away from any debris or contamination, and prevents extraneous spray from contacting other body parts. The goggle-like dispenser assures that most of the medication is absorbed in the eye and surrounding area, and prevents medicament from being dispersed into the atmosphere, which is wasteful and renders medication dosing inaccurate.

The goggle-like dispenser is, according various embodiments of the invention, a fully mechanical and requires no electrical power source or components.

The goggle-like dispenser directs a steady mist gently blanketing and settling on the surface of the eye. A peripheral portion is targeted at a non-perpendicular angle so that the mist does not interfere with vision or irritate the eye. This potential technology overcomes disadvantages of directing a fluid stream directly at the eye.

The goggle-like dispenser discloses an atomization mechanism which provides a non-eye-impinging droplets cloud, unlike “inkjet” type dispensing mechanisms that operate through a thermal-expansion principle. Moreover, the current technology overcomes the problems inherent in heating systems which include degradation of medicaments etc.

The goggle-like dispenser teaches in one of its embodiments, control of droplet size. The dispenser provides predictable droplets size due to predetermined and accurate action of the Venturi pump and described herein below.

Controlling droplet direction is important as a mechanism to effectively deliver the drug to the eye and reduce the chances of impinging the eye. The present invention uses, inter alia, a nozzle less Venturi to spray the mist, so that the properties of the spray can be controlled. The dispenser's spray is possibly not linear; it may have, according to various embodiments, a certain dispersion that can be controlled with the parameters of the nozzle. The size and shape of the spray may be also controlled. The nozzle can be altered to produce a spray with an elliptical cross-section, for example, to better cover the surface of the eye. This technology avoids randomized large droplets colliding in turbulent air to form even larger droplets.

Droplet velocity is an important factor because of its contribution to momentum. It is also more widely distributed than droplet size. The goggle-like dispenser provides very low mass droplets with very short ballistic trajectories at velocities which produce droplets of low momentum. Such low momentum droplets of high surface-to-volume ratios are designed to transfer very low impact to the eye and deliver maximum dosage.

Controlling exposure time to the fluid is a way of controlling the dose. The goggle-like dispenser provides, according to one embodiment of the invention, a single-action mechanism which actuates both mist production and ejection, and is an “idiot proof” operating system. Moreover, the unpleasant experience of having a medication applied directly to the eye is minimized due to the gentleness of the application and the brief duration of the procedure (seconds).

The goggle-like dispenser discloses ability to control the exposure time through droplet size, velocity and realizing time. Via controlling the exposure time one controls the absorption of the active compound into the eye. The goggle-like dispenser enables an exposure time measure in seconds.

The dispenser defined and described in the present invention avoids or at least reduces the risk of medication overdose. For example, beta-blocker overdose is known to be potentially life-threatening. One of the treatments for glaucoma is with a class of medications known as beta-blockers (e.g. timolol) that are applied twice a day. Due to systemic absorption via the nasopharynx through the nasolacrimal duct, such drug can cause cardiac arrhythmias and pulmonary problems (asthma, chronic obstructive pulmonary disease). In patients treated systemically with beta-blockers (e.g. commercially available propranolol, atenolol and other medicaments) and already have a near-toxic level in their system, treatment with the hereby presented dispenser minimizes the risk of overdose.

The novel single action mechanism of the goggle-like dispenser, as defined in various embodiments of the present invention, prevents accidental over-dosing, since a precise portion of the medicament is delivered each time. Moreover, the dispensing method precludes production of large coalesced droplets landing on the surface of the eye providing, at least localized, over dose of irritating medicaments and solvents.

The goggle-like dispenser seals, according to one embodiment of the invention, the eye away from any debris or contamination, and prevents extraneous spray from contacting other body parts. The goggle-like dispenser assures that most of the medication is absorbed in the eye and surrounding area, and prevents medicaments to disperse into the environment which is both wasteful of active medicament and also render accurate dosing impossible. The current technology further overcomes spectacles-frame type solutions not isolated from the environment; goggle-like mechanisms, with intake ports for air circulation; and very large air volume type solutions.

The goggle-like dispenser provides, according one embodiment of the invention, a non-circulating spray tangential to the eye, of small droplets within a small hermetically confined volume, thus any given droplets pass a relatively large surface of the eye, at a very oblique angle. Hence, the unique combination of a small hermetically confined goggle, with a side-directed spray orifice and non-circulating mist avoids impinging and directly striking the eye by droplets.

According to another embodiment of the invention, the Venturi nozzle dispensing apparatus having a lumen-within-lumen configuration. At least one inner lumen is accommodated within at least one outer lumen, either in a concentric or eccentric manner. The main longitudinal axis of the at least one inner lumen is either parallel or otherwise tilted at an angle θ in respect to the longitudinal axis of the at least one outer lumen

According to another embodiment of the invention, the Venturi nozzle dispensing apparatus as defined in any of the above is utilized for both mixing and dispensing one or more fluids. It is in the scope of the invention wherein the term ‘fluid’ refers in a non-limiting manner to liquids, gases, aerosols, solid-state fine particles (e.g., in a nano- or micrometric scale) or any mixtures thereof.

The term “fluid” also refers to pre-treated fluids and/or post-treated fluids, such as humidified or dried fluids, electrically charged fluid etc. The term also refers to slurry, a colloid, an emulsion and other mixtures of fluids and medications. The terms fluid and medicaments further interchangeably refer to a fluid or medicament which is a product, or a by-product, of a chemical or a physical reaction, e.g., a reaction which instantaneously provided upon admixing two or more raw materials, catalysts, or other compounds.

According to another embodiment of the invention, the Venturi nozzle dispensing apparatus as defined in any of the above is utilized for a multiphase (e.g., bi-phase) dispensing of fluids and medicaments thereof. In one case, microcontainers (e.g., bullet-like compartments) as defined above are used. In one possible arrangement, each microcontainer comprises either a mixture or multiple separate phases provided in a compartmentablity achieved by membrane-like barriers. The at least one liquid and at least one gas are stored in this microcontainer above atmospheric pressure (e.g., respectively low pressure of about 3 to about 4 atm, higher pressure of about 10 to about 50 atm etc.). When the microcontainer is pierced or otherwise exposed to external pressure, gas volume expands (about 3 to about 4 times) while the liquids remain in their initial volume. The facilitated flow of the expending gas mixes the fluid and simultaneously carries it along a preset dispensing pathway.

In contrast to a dispenser which comprises nozzles, i.e., where the characteristics of the fluid's flow, and a result, the dispensed aerosol, is determined mainly by the nozzle type, shape and other parameters; the fluid's flow and the dispensed aerosol provided by the Venturi nozzle dispensing apparatus as defined above, is not determined in inner sections of the apparatus but by the dispensing outlet and along aerosol tracts located downstream in respect to the outlet. The location, shape and size of the outlet are the main parameters defining and regulating the fluid flow and aerosol dispensed thereof.

It is hence in the scope of the invention wherein the dispenser is Venturi nozzle, and the characteristics of the dispensed aerosol are determined solely by the outlet, including droplets size and distribution, flow flux, beam characterization and direction. Hence for example, the fluid flow characteristics (e.g., rotational and three-dimensional vectors of the fluid column) adjacent to the outlet, along and around the main longitudinal axis of the dispenser, are provided by the combination of the Bernoulli principle within the aforesaid W-junction and the Venturi effect of the outlet.

It is alternatively in the scope of the invention wherein the outlet is in fluid communication with an external mechanisms, located downstream to the outlet. Such a mechanism is selected in a non-limiting manner from a group consisting of either conical or cylindrical protrusions, static flow guides (baffles etc), dynamic dispensing mechanisms such as fans, turbulents etc.

It is alternatively in the scope of the invention wherein a nozzle unit is positioned upstream in respect to the outlet, to provide the fluid flow with a few characteristics, when the outlet and potential external mechanism provides the fluid flow and aerosol dispensed thereof its unique characterization.

It is alternatively in the scope of the invention wherein a pressurized two-phase or other multi-phase reservoir is utilized. The reservoir consists of at least one medication and carrier, such as N₂ or air. The liquid medicament phase and the gaseous carrier phase are mixed in the reservoir in a predetermined set of conditions (pressure, volume etc). Upon initiating facilitated flow of the mixture, via a specially crafted orifice (nozzle) or a Venturi nozzle system, a spray characterized by a given droplet size, distribution and velocity is provided. Alternatively, according to yet another embodiment of the invention, foam, a slug flow, an annular flow and/or a stratified flow is potentially obtainable.

The system and method defined above alternatively provides a gas-less, Venturi-effect driven ad-hoc gas-liquid admixture. Alternatively or additionally, high speed liquid launch is provided useful. The liquid stream is launched in a nozzle-velocity ranging from about ×10 to about ×100 msec into the confined volume surrounding the patient's eye to be treated. The energy and force balance between the surface tension and the drag forces causes rapid breakdown of the initial drop into even smaller droplets until equilibrium is reached, such as about 1 to 10 micrometer size droplets are obtained. It is well in the scope of the invention wherein the energy source for the acceleration of the medicament liquid phase is provided by a multiplier cylinder, a spring-activated step function generator or other commercially available mechanisms.

It is in the scope of the present invention wherein the goggle-like dispenser is provided useful for treating the eye, especially, yet not exclusively for treating pathologies of the eye and adnexa of the eye, e.g., conjunctiva, eyelashes, tear ducts, and the bony orbit; treating eye allergy, e.g., by dispensing allergy medications that are selected in a non-limiting manner from a group consisting of ketorolac tromethane, loteprednol etabonate, epinastine hydrochloride, emedastine difumarate, levocabastine hydrochloride, azelastine hydrochloride, olopatadine hydrochloride, ketotifen fumarate, potassium pemirolast, sodium nedocromil, lodoxamide tromethamine and sodium cromolyn; treating infections, e.g., by dispensing antibiotic medications, such as aminoglycosides, ciprofloxacin, ofloxacin, levofloxacin, moxyfloxacin, gatifloxacin, tobramycin, gentamycin, erythromycin, and bacitracin, polymyxin B combinations, such as polymyxin B and trimethoprim, polymyxin B and bacitracin, polymixin B and neomycin and gramycin. The goggle-like dispenser is provided useful for treating the eye and dispensing corticosteroid medications, such those that are selected from a group consisting of prednisolone acetate, hydrocortisone, dexamethosone, fluorometholone, prednisolone acetate, dexamethosone, prednisolone sodium phosphate. The goggle-like dispenser is also provided useful for treating the eye and dispensing corticosteroid/antibiotic combinations, such as prednisolone acetate, sodium sulfacetaqmide, hydrocortisone dexamethosone fluorometholone, prednisolone acetate, dexamethosone, prednisolone sodium phosphate combined with any of the group consisting of sulfacetamide, neomycin, polymyxin B, neomycin, gentamycin, and tobramycin.

The goggle-like dispenser is provided useful for treating glaucoma by dispensing the eye glaucoma medications, such as levobunolol hydrochloride, timolol hemihydrate, betaxolol hydrochloride, timolol maleate, timolol maleate, timolol hemihydrate, bimatoprost, travoprost, latanoprost, unoprostone, brimonidine, apraclonidine, brinzolamide, dorzikanude, and dorzolamide/timolol. It is a further object of the present invention wherein the glaucoma medication is selected from a group consisting of beta blockers, prostaglandin analogs, docosanoid compounds, alpha agonists, carbonic anhydrase inhibitors, and combinations thereof.

The goggle-like dispenser is provided useful for treating dry eye by dispensing the eye dry eye medicaments, such as hydroxypropyl methylcellulose, carboxylmethylcellulose, polyvinyl alcohol, glycerine, polyethylene glycol, propylene glycol, castor oil, polysorbate-80, and mineral oil.

The goggle-like dispenser is provided useful for contact lens rewetting drops, eye ointments, and eye gels.

It is in the scope of the present invention wherein the goggle-like dispenser is adapted to dispensed a medicament or medicaments selected, in a non-limiting manner, from a group consisting of saline-containing solutions, lubricants-containing solutions, steroids (e.g., dexamethasone), antihistamines, sympathomimetics, beta receptor blockers, parasympathomimetics (e.g., pilocarpine), parasympatholytics (e.g., tropicamide or atropine), prostaglandins, non-steroidal anti-inflammatory drugs or topical anesthetics, pigments, dyes, biocides, antibiotics (e.g., bactericides, anti-viral agents, fungicides) or any combination thereof. Additionally or alternatively, the medicament or medicaments are selected, in a non-limiting manner, from a group consisting of antihistamines, decongestants, nasal steroids, corticosteroids, mornetasone furoate, fluticasone furoate, oxymetazoline, carbon dioxide, saline solution, oxymetazoline hydrochloride, glucocorticosteroid, diphenhydramine, prednisone, epinastine, olopatadine, itraconazole, ciclesonide, adrenalin, L-asparaginase-methotrexate-dexamethasone, systemic antibiotics, antidepressants, antifungal medications, anti-inflammatory medications, antiviral medications, analgesics, anticonvulsants, and any other formulations intended to breach the blood-brain barrier.

Additionally or alternatively, the medicament or medicaments are selected, in a non-limiting manner, from a group consisting corticosteroids, beta-2 agonists, leukotriene modifiers, anti-leukotrienes, cromolyn, nedocromil, theophylline, immunotherapeutic agents, anti-IgE monoclonal antibodies, insulin, albuterol, pirbuterol, levalbuterol, bitolterol, ipratropium (atrovent), prednisone, prednisolone, methylprednisolone, hydrocortisone, and serevent; allergy medications selected from a group consisting of ketorolac tromethane, loteprednol etabonate, epinastine hydro chloride, emedastine difumarate, levocabastine hydrochloride, azelastine hydrochloride, olopatadine hydrochloride, ketotifen fumarate, potassium pemirolast, sodium nedocromil, lodoxamide tromethamine and sodium cromolyn, ciprofloxacin, ofloxacin, levofloxacin, moxyfloxacin, gatifloxacin, tobramycin, gentamycin, erythromycin, aminoglycosides, bacitracin, polymyxin B, any combination of Polymyxin B and trimethoprim, any combination of polymyxin B and bacitracin, and any combination of polymixin B, prednisolone acetate, hydrocortisone, dexamethosone, fluorometholone, prednisolone acetate, dexamethosone, prednisolone sodium phosphate, neomycin and gramycin.

Additionally or alternatively, the medicament or medicaments are selected, in a non-limiting manner, from a group consisting corticosteroid medication consisting of prednisolone acetate, sodium sulfacetaqmide, hydrocortisone dexamethosone fluorometholone, prednisolone acetate, dexamethosone, prednisolone sodium phosphate combined with any of the group consisting of sulfacetamide, neomycin, polymyxin B, neomycin, gentamycin, and tobramycin, levobunolol hydrochloride, timolol hemihydrate, betaxolol hydrochloride, timolol maleate, timolol maleate, timolol hemihydrate, bimatoprost, travoprost, latanoprost, unoprostone, brimonidine, apraclonidine, brinzolamide, dorzikanude, and dorzolamide/timolol, beta blockers, prostaglandin analogs, docosanoid compounds, alpha agonists, carbonic anhydrase inhibitors, hydroxypropyl methylcellulose, carboxyl methylcellulose, polyvinyl alcohol, glycerine, polyethylene glycol, propylene glycol, castor oil, polysorbate-80, mineral oil, contact lens rewetting drops, eye ointments, and eye gels and combinations thereof.

Additionally or alternatively, the medicament or medicaments are selected, in a non-limiting manner, from a group consisting antihistamines, decongestants, nasal steroids, corticosteroids, mornetasone furoate, fluticasone furoate, oxymetazoline, carbon dioxide, saline solution, oxymetazoline hydrochloride, glucocorticosteroid, diphenhydramine, prednisone, epinastine, olopatadine, itraconazole, ciclesonide, adrenalin, L-asparaginase-methotrexate-dexamethasone, systemic antibiotics, antidepressants, antifungal medications, anti-inflammatory medications, antiviral medications, analgesics, anticonvulsants, and any other formulations intended to breach the blood-brain barrier.

A method of dispensing ophthalmic medicaments, comprises: providing a goggle-like dispenser of ophthalmologic medicaments, comprising: a single goggle held over the eye, comprising: a seal to prevent escape of the medicament mist within a confined volume surrounding the patient's eye; a mechanism for storing and mechanically delivering preservative-free medicaments; and a dispenser to dispense a steady mist flow consisting of droplet sizes on the range of approximately less than 50 microns, said dispenser having an outlet, in a fluid connection with at least one Bernoulli-type junction of multiple W conduits, W is an integer equal to or bigger than two; filling said container with said ophthalmic medicament; placing said device on the patient; and, facilitating a flow of a mist or vapor medicament into the confined volume defined between the patient's eye and the lens; wherein said facilitating is performed in a manner that gently blanket and settle on the surface of the eye and that do not interfere with vision or irritate the eye.

A method of dispensing ophthalmic medicaments, comprises: providing a goggle-like dispenser of ophthalmologic medicaments, comprising: a single goggle held over the eye, comprising: a seal to prevent escape of the medicament mist within a confined volume surrounding the patient's eye; a mechanism for storing and mechanically delivering preservative-free medicaments; and a dispenser to dispense a steady mist consisting of droplet sizes on the range of approximately less than 50 microns; filling said container with said ophthalmic medicament; placing said device on the patient; and, facilitating a flow of a mist or vapor medicament into the confined volume defined between the patient's eye and the lens; wherein said facilitating is performed in a manner that gently blanket and settle on the surface of the eye and that do not interfere with vision or irritate the eye.

It is in the scope of the invention wherein a method dispensing ophthalmic medicaments is provided. The method comprises steps selected from a group consisting, inter alia, of obtaining an either single-use (at least partially disposable); placing at least medicament within a container or microcontainer, or a plurality of containers and microcontainers; is the device comprises a wing, swiveling or otherwise positioning the wing to its open configuration; introducing the goggle-like dispenser to the eye or to other organ to be treated; if relevant, adjusting the dosing valve to a preset parameter appropriate to the medicament to be dispensed and/or the treating protocol; pressing the control valve such as a cloud of mist is flow in a direct and a non-eye-impinging manner and gently coating the organ to be treated; removing the device from the organ, and possibly, either manually or automatically, flushing the device; and finally, swiveling or otherwise positioning the wing to its normal close configuration. Other steps are possible, such as introducing a new microcontainer within a microcontainer-housing and connector thereof; replacing disposable mechanisms of the device prior to their use etc.

It is also in the scope of the invention to disclose methods for treating humans and animals. More specifically, yet in a non-limiting manner, the methods are adapted to treat disorders of sclera, cornea, iris and ciliary body, such as Scleritis, Keratitis, Corneal ulcer or Corneal abrasion, Snow blindness or Arc eye, Thygeson's superficial punctate keratopathy, Corneal neovascularization, Fuchs' dystrophy, Keratoconus, Keratoconjunctivitis sicca, ritis, Uveitis etc. Alternatively or additionally, yet in a non-limiting manner, the methods are adapted to treat disorders of lens, such as Cataract. Alternatively or additionally, yet in a non-limiting manner, the methods are adapted to treat disorders of choroid and retina, Retinal detachment, Retinoschisis, Hypertensive retinopathy, Diabetic retinopathy, Retinopathy, Retinopathy of prematurity, Age-related macular degeneration, Macular degeneration, Retinitis pigmentosa Macular edema etc. Alternatively or additionally, yet in a non-limiting manner, the methods are adapted to treat Glaucoma. Alternatively or additionally, yet in a non-limiting manner, the methods are adapted to treat disorders of vitreous body and globe, such as Floaters. Alternatively or additionally, yet in a non-limiting manner, the methods are adapted to treat disorders of optic nerve and visual pathways, such as Leber's hereditary optic neuropathy. Alternatively or additionally, yet in a non-limiting manner, the methods are adapted to treat disorders of ocular muscles, binocular movement, accommodation and refraction, such as Strabismus, Ophthalmoparesis, Progressive external ophthalmoplegia, Esotropia, Exotropia etc. Alternatively or additionally, yet in a non-limiting manner, the methods are adapted to treat disorders of refraction and accommodation, such as Hypermetropia, Myopia, Astigmatism, Anisometropia, Presbyopia etc. Alternatively or additionally, yet in a non-limiting manner, the methods are adapted to treat disorders of accommodation, such as Internal ophthalmoplegia, Visual disturbances and blindness, Amblyopia (lazy eye), Leber's congenital amaurosis, Scotoma, Color blindness, Achromatopsia or Maskun, Nyctalopia, River blindness, micro-opthalmia or coloboma. Alternatively or additionally, yet in a non-limiting manner, the methods are adapted to treat other disorders of eye and adnexa, such as Red eye, Argyll Robertson pupil, Keratomycosis, Xerophthalmia, Aniridia etc. Alternatively or additionally, yet in a non-limiting manner, the methods as defined in any of the above are further or alternatively adapted to treat disorders located in human's or animal's organs, other then the eye, such as the ear, the nose, the mouth, the throat, the vagina, the rectal cavity, the urological lower tracks, the skin, hair or fur etc.

All patents, patent applications, and published references cited herein are hereby incorporated by reference in their entirety. It will be appreciated that several of the above-disclosed and other features and functions, or alternatives thereof, may be desirably combined into many other different systems or applications. Various presently unforeseen or unanticipated alternatives, modifications, variations, or improvements therein may be subsequently made by those skilled in the art.

1-32. (canceled)

33. A goggle-like dispenser of ophthalmologic medicaments comprising: a single goggle held over the eye comprising: a seal to prevent escape of the medicament mist within a confined volume surrounding the patient's eye; a mechanism for storing and mechanically delivering preservative-free medicaments; and a dispenser to dispense a steady mist having droplet sizes on the range of approximately less than 50 microns that gently blanket and settle on the surface of the eye and that do not interfere with vision or irritate the eye.

34. The goggle-like dispenser of claim 33, further comprising:
at least one container adapted for accommodating ophthalmologic medicaments;
at least one nozzle placed in front of a peripheral portion of a patient's eye slot, provided in a fluid interconnection with said container; said nozzle mechanically secured to said dispenser body and adapted to provide said medicament mist flow;
wherein said nozzle is adapted to laterally provide said mist flow within said confined volume in a direct and-non-eye-impinging manner.

35. The goggle-like dispenser of claim 34, wherein said at least one container is incorporated within said frame or within said lens.

36. The goggle-like dispenser of claim 34, wherein said nozzle is mechanically secured to said frame or to said lens.

37. The goggle-like dispenser of claim 34, wherein said medicament is accommodated in said container in either a highly pressurized nitrogen-free state or-non-pressurized manner.

38. The goggle-like dispenser of claim 33, wherein said medicament mist is preservative-free.

39. The goggle-like dispenser of claim 38, wherein at least one of the following is being held true (a) said control mechanism is provided with a one-way dosing valve; (b) said control mechanism regulates or meters one or more fluid's parameters selected from a group consisting of dispensing-time protocol, droplet volume, fluid flux, droplet weight, batch unit, an any combination thereof; and any combination thereof.

40. The goggle-like dispenser of claim 33, wherein said medicament mist is dispensed in such a manner that it remains in a dispersed state for a preset time scale, said time scale is between about 0.5 sec to about 5 sec, between about 5 sec to about 40 sec, between about 10 to about 50 sec, or between about 15 to about 60 sec.

41. The goggle-like dispenser of claim 33, further comprising a medicament heater and/or cooler.

42. The goggle-like dispenser of claim 33, wherein the at least one container is a plurality of microcontainers, in particular disposable microcontainers; and said microcontainers are individually connectible in fluid-connection with said nozzle; further wherein said microcontainers are fed by a feeding mechanism selected from the group consisting of a revolving nosepiece, a cartridge, a magazine, a clip, and any combination thereof.

43. The goggle-like dispenser of claim 42, comprising a connector adapted to consecutively or concurrently open said microcontainers and feed corresponding doses of said ophthalmic medicament into said nozzles.

44. The goggle-like dispenser of claim 33, further comprising at least one protective cover; adapted to be folded over the eyecup in between uses to protect the eyecup from contamination from the outside environment; further wherein said protective cover is a form-fitting wing.

45. A goggle-like Venturi nozzle dispenser of ophthalmologic medicaments comprising: a single goggle held over the eye, comprising: a seal to prevent escape of the medicament mist within a confined volume surrounding the patient's eye; a mechanism for storing and mechanically delivering preservative-free medicaments; and a dispenser to dispense a steady mist flow having an outlet, in a fluid connection with at least one Bernoulli-type junction of multiple W conduits, W is an integer equal to or bigger than two; said mist having droplet sizes on the range of approximately less than 50 microns that gently blanket and settle on the surface of the eye and that do not interfere with vision or irritate the eye.

46. The goggle-like Venturi nozzle dispenser of claim 45, wherein at least one of the following is being held true: (a) said multiple W conduits provides a multiple conduits junction, said junction is selected from a group consisting of L-type, Y-type, T-type and X-type; (b) the angle between the main longitudinal axis of two conduits is angle theta q, and wherein q is more than 0°; (c) angle q is about 90°, and especially wherein q ranges between about 75° to about 85°; and any combination thereof.

47. The goggle-like Venturi nozzle dispenser of claim 46, wherein at least one of the following is being held true (a) said at least one container is incorporated within said frame or within said lens; (b) said fluid outlet is mechanically secured to said frame or to said lens; (c) wherein said medicament is accommodated in said container in either a highly pressurized nitrogen-free state or non-pressurized manner; (d) said medicament mist is preservative-free; (e) said goggle-like Venturi nozzle dispenser further comprising a control mechanism adapted to control doses of said medicament; and any combination thereof.

48. The goggle-like Venturi nozzle dispenser of claim 47, wherein said control mechanism is provided with a one-way dosing valve.

49. The goggle-like Venturi nozzle dispenser of claim 48, wherein said dosing is provided by one or more of a group consisting of predetermined time protocol, volume, flux, weight, batch unit, number of bursts, and any combination thereof.

50. The goggle-like Venturi nozzle dispenser of claim 47, wherein said medicament mist is dispensed in such a manner that it remains non-condensable matter for a preset time scale, said time scale is between about 1 to about 5 sec, between 5 sec to about 40 sec, between about 10 to about 50 sec, or between about 15 to about 60 sec.

51. The goggle-like Venturi nozzle dispenser of claim 47, further comprising at least one selected from a group consisting of (a) medicament heater and/or cooler; (b) protective cover, especially a form-fitting wing; said protective cover permanently or temporarily attached to said lens or dispenser body by mechanical attachment such as a hinge or equivalent, and effectively forming a seal when closed; and any combination thereof.

52. The goggle-like Venturi nozzle dispenser of claim 47, wherein the mechanism for storing of the medicaments is a plurality of microcontainers; said microcontainers are individually connectible in fluid-connection with said nozzle.

53. A method of dispensing ophthalmic medicaments comprising:
providing a goggle-like dispenser of ophthalmologic medicaments, comprising a single goggle held over the eye comprising: a seal to prevent escape of the medicament mist within a confined volume surrounding the patient's eye; a mechanism for storing and mechanically delivering preservative-free medicaments; and a dispenser to dispense a steady mist flow consisting of droplet sizes on the range of approximately less than 50 microns, said dispenser having an outlet, in a fluid connection with at least one Bernoulli-type junction of multiple W conduits, W is an integer equal to or bigger than two;
filling said container with said ophthalmic medicament;
placing said device on the patient; and,
facilitating a flow of a mist or vapor medicament into the confined volume defined between the patient's eye and the lens;
wherein said facilitating is performed in a manner that gently blanket and settle on the surface of the eye and that do not interfere with vision or irritate the eye.

54. A method of dispensing ophthalmic medicaments comprising:
providing a goggle-like dispenser of ophthalmologic medicaments, comprising a single goggle held over the eye comprising: a seal to prevent escape of the medicament mist within a confined volume surrounding the patient's eye; a mechanism for storing and mechanically delivering preservative-free medicaments; and a dispenser to dispense a steady mist consisting of droplet sizes on the range of approximately less than 50 microns;
filling said container with said ophthalmic medicament;
placing said device on the patient; and,
facilitating a flow of a mist or vapor medicament into the confined volume defined between the patient's eye and the lens;
wherein said facilitating is performed in a manner that gently blanket and settle on the surface of the eye and that do not interfere with vision or irritate the eye.
